(19) United States
(12) Patent Application Publication
(10) Pub. No.: US 2011/0208136 A1
(43) Pub. Date: Aug. 25, 2011

(54) METHOD FOR APPLYING A LIQUID MEDICINAL TREATMENT MEDIUM TO AN AREA TO BE TREATED AND MEDICINAL PRODUCT

(76) Inventors: Horst Sollingen, Feldkirchen (DE); Jahann Beil, Viechtach (DE)

(21) Appl. No.: 12/733,888

(22) PCT Filed: Sep. 25, 2008
(86) PCT No.: PCT/DE2008/001576

§ 371 Date: May 4, 2011

(30) Foreign Application Priority Data
Sep. 27, 2007 (DE) 10 2007 046 600.7

(51) Int. Cl. A61M 35/00

(52) U.S. Cl. 604/290; 604/310

(57) ABSTRACT

The invention relates to a method for the targeted application of a liquid medicinal treatment medium for topical treatment of a human or animal body in an area to be treated. The treatment medium is applied using at least one pin-like applicator.

### BACKGROUND OF THE INVENTION

The invention relates to a method for applying a liquid medicinal treatment medium to an area to be treated and a medicinal product.

It is an object of the invention is to provide a method which enables an easy, well-dosed and above all also precisely localized application of a liquid medicinal treatment medium to a treatment area (skin or tissue) of a human and/or animal body.

“Medicinal treatment medium” according to the invention is understood to be a medium which in solution contains at least one medicinal substance, for example for disinfection, for treating chronic wounds, such as Ulcus cruris or Decubitus, and also for other medicinal indications, for example for treating calluses, warts, acne, herpes, onychomycosis, fungal infections of the head, changes in pigment or pathological proliferations of the skin and mucous membrane, respectively by external application.

The solution containing the at least one active substance is for example an oily solution or an aqueous alcoholic solution, which is selected so that the required flow of the treatment medium from the tubular or sleeve-shaped container through the application tip on its end used for application (application end) is ensured in particular also without clogging of the pores and/or channels within the application tip, the solution however ensuring the required stability of the treatment medium, in particular also with respect to the physical and/or chemical properties. The applicator used is for example the size of a normal felt-tip pen or marker. It is also possible, however, to select the size of the applicator and/or the volume of the interior or reservoir of this applicator used for holding the treatment medium so that it is so small that the treatment medium is sufficient only for a single treatment or only for a very limited number of treatments.

Application of the treatment medium generally is effected by brushing over the area to be treated with the application tip or with its application end, the application tip preferably being made of a material that is suitable for the controlled and dosed passage of the treatment medium, for example of a porous material, or of a sintered material, e.g. of a sintered plastic.

In further embodiments of the invention, the method according to the invention and/or the medicinal product according to the invention are designed for example so that

the treatment medium contains at least one active substance in an oily solution,
and/or
the oily solution of the treatment medium contains at least one mineral oil and/or at least one derivative of a mineral oil,
and/or
the mineral oil is paraffin oil and/or silicone oil,
and/or
the treatment medium or its solution contains at least one vegetable oil and/or oil product which is obtained from at least one vegetable oil by ester interchange, partial reduction, etherification and/or partial synthesis,
and/or
sunflower oil and/or other vegetable oils listed in the pharmacopeia are used as the vegetable oil,
and/or
glycerol ester or other esters of fatty acids with fatty alcohols, fatty alcohols and/or their ethers and esters are used as the oil product, also in varying combinations and in varying concentrations,
and/or
the oily solution contains at least one additive in the form of tensides, preferably with a content of approximately 0.1-4.0% by weight relative to the total weight of the solution, and/or in the form of waxes, preferably with a content of approximately 0.1-1.0% by weight relative to the total weight of the solution, and/or fragrant oils and/or colorants,
and/or
the treatment medium contains the at least one medicinal substance in an aqueous alcoholic solution,
and/or
the aqueous alcoholic solution contains monohydric or polyhydric alcohols, e.g. ethanol, glycerol and/or polymer alcohols and/or isopropanol and/or propylene glycol and/or PEG and/or their esters and/or ethers,
and/or
the aqueous solution contains ethanol and/or isopropanol in a concentration of approximately 5.0-70% by weight relative to the total weight of the solution,
and/or
the aqueous alcoholic solution contains the polyhydric, saturated and/or unsaturated alcohols and polymer alcohols, such as propylene glycol and/or their esters and/or ethers in a concentration of approximately 2.0-40% by weight relative to the total weight of the solution,
and/or
the aqueous alcoholic solution contains at least one medicinal substance in liposomal and/or nanosomal encapsulation,
and/or
the aqueous alcoholic solution contains a film-forming component, preferably a polymer film-forming component,
and/or
ethyl acetate, preferably in a concentration of approximately 5.0-40% by weight and/or acetone, preferably in a concentration of approximately 5.0-40% by weight relative to the total weight of the solution, respectively, is used as an additional solvent,
and/or
polymers and/or co- or cross-polymers, such as polymethacrylate, preferably also in chemically modified form, are used as the film-forming component,
and/or
the aqueous alcoholic solution contains additives in the form of tensides, preferably in a concentration of approximately 0.5-5% by weight relative to the total weight of the solution, and/or fatty alcohols and/or their esters and ethers, preferably in a concentration of approximately 0.5-4.0% by weight relative to the total weight of the solution,
and/or
polysobat 80 and/or octyl dodecanol is used as an additive,
and/or
the aqueous alcoholic solution contains additives in the form of thickeners based on cellulose and/or starch, for example oligosaccharide and/or polyacrylate, preferably in a concentration of approximately 0.1-0.4% by weight relative to the total weight of the solution,
and/or
the solution contains fragrant oils and/or colorants,
and/or
at least one medicinal substance for treating calluses, warts, acne, herpes, onychomycosis, fungal infections on the head, changes in pigment or pathological proliferations of the skin is contained,
and/or
at least one medicinal substance for treating chronic wounds, such as Ulcus cruris or Decubitus is contained,
and/or
the treatment medium in the solution contains at least one enzymatic and/or antimicrobial substance,
and/or
the application tip is made of a porous material, for example of a porous sintered material, e.g. of a sintered polymer material,
and/or
the application tip is designed with reduced hardness on its end used to apply the treatment medium to the skin,
and/or
the application tip exhibits reduced porosity on its end (5.1) used to apply the treatment medium to the skin,
and/or
the application tip is made of PE, PP, PVDF, PTFE and/or EVA,
and/or
the container has a sleeve-shaped or tubular design,
and that the foregoing characteristics can be used singly or in any combination.

Further embodiments, advantages and applications of the invention are disclosed in the following description of exemplary embodiments and the drawings. All characteristics described and/or pictorially represented, alone or in any combination, are subject matter of the invention.

### BRIEF DESCRIPTION OF THE DRAWINGS

The invention is described below in detail based on exemplary embodiments with reference to the drawings, in which:

FIG. 1 shows in an exploded view the elements of a device for applying a liquid medicinally effective treatment medium according to the invention;

FIG. 2 shows the device in FIG. 1 in assembled state; and

FIGS. 3 and 4 show views as in FIGS. 1 and 2 of a further embodiment.

### DETAILED DESCRIPTION OF THE INVENTION

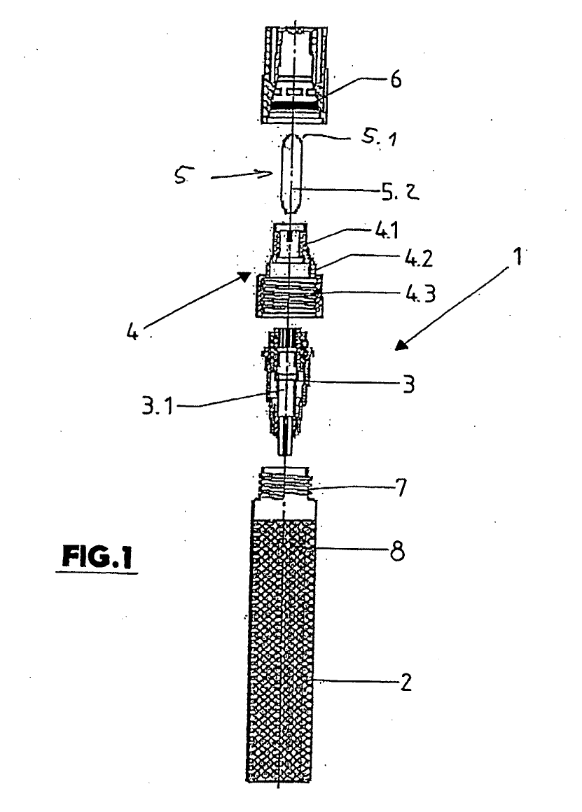

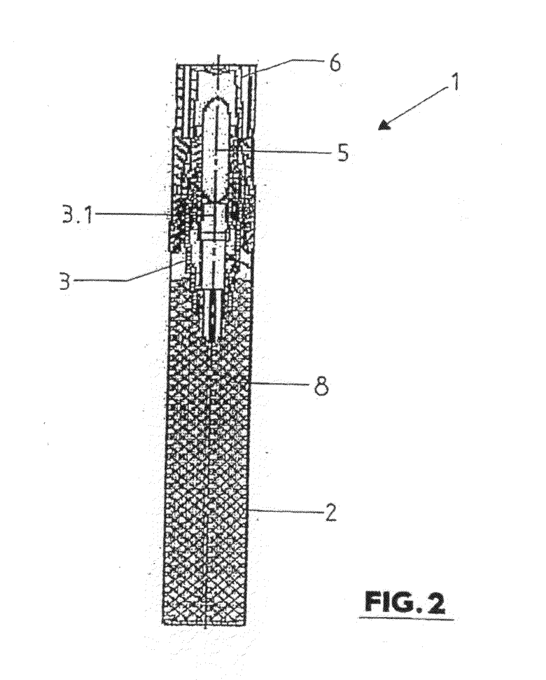

The device (applicator) generally designated 1 in FIGS. 1 and 2 consists of a sleeve-shaped or tubular container 2 made of a suitable material, for example of plastic, glass and/or metal, which is open only on one end, i.e. in the embodiment depicted in FIG. 1 on its upper end for holding the treatment medium, of an insert 3 containing a valve or dosing pump 3.1, of a sleeve-shaped mouth piece 4 with sections 4.1, 4.2 and 4.3 with different inner and outer diameters, of an application tip 5 and of a cap 6, which is manufactured as an injected molded part from plastic, as are at least parts of the insert 3 and the mouth piece 4.

The application tip 5 is made of a porous, liquid-conducting material and is designed for example so that its end protruding from the mouth piece 4 in assembled state of the applicator 1, i.e. on the end 5.1 (application end) there used for application of the treatment medium, is softer than in the remaining area or shank 5.2. Furthermore, the porosity of the application tip 5 is such that it has a reduced pore size on its end 5.1 than in the shank 5.2, i.e. the pore size at the end 5.1 is for example between approximately 18 and 30 μm and in the area of the shank 5.2 is between approximately 40 and 60 μm. Due to the coarser pore structure and the harder design of the application tip 5 on the shank 5.2 the supply of the liquid treatment medium at the end 5.1 is improved and at the same time due to the harder design of the shank 5.1 the mechanical properties of the application tip are significantly better adapted to the requirements for automatic assembly.

Suitable materials for the application tip 5 are for example porous, liquid conducting materials, such as porous plastic materials, porous ceramic and also felt-like or fleece-like materials. Preferably the application tip is made of a sintered material, for example of a sintered polymer material, e.g. PE, PP, PVDF, PTFE or EVA. Due to the hydrophobic properties of the polymer materials it is advantageous to treat these materials with a hydrophilic treatment.

Furthermore, it can be advantageous to provide the application tip 5 on its peripheral surface with at least one aeration or ventilation groove extending over a majority or over the entire length of the application tip 5, to prevent dripping or leaking of the applicator 1 especially as a result of thermal effects. Furthermore, the application tip 5 is preferably tapered at least on its end 5.1, or for example on both ends, namely for example with a rounded tip.

In assembled state the insert 3 is inserted into the opening of the container 2 and secured there in a suitable manner, e.g. by screwing of the section 4.3 of the mouth piece 4 provided with the internal thread, so that the insert 3 is held at the opening of the container 2 and sealing this opening, extends with a sleeve-shaped inlet of its dosing pump 3.1 into the interior of the container 2 or into the liquid treatment medium 8 stored there and ends with the outlet of its dosing pump 3.1 in the interior of the section 4.2 of the mouth piece 4. The application tip 5 is inserted with its shank 5.2 into the mouth piece 4, namely so that it protrudes with its end 5.1 over the end depicted in FIGS. 1 and 2 as the upper end of the mouth piece 4 and with its lower end into the interior of the section 4.2 or into the upper end of the insert 3 held there and axially opposes the dosing pump directly. By axial movement of the application tip 5 against the effect of a recoil spring (not depicted) provided for example in the dosing pump 3.1, the dosing pump 3.1 can be actuated, namely for removing a dosed quantity of the treatment medium 8 from the container 2 and for supplying this quantity to the application tip 5.

The dosing pump 3.1 can also have a different design, namely so that it is actuated by exerting radial mechanical pressure on the mouth piece 4 and/or on the container 2, which is for example elastically deformable. Furthermore, it is also possible to provide instead of the dosing pump 3.1 a dosing valve, which opens upon exertion of the radial pressure on the applicator 1, in particular on the mouth piece and/or on the elastic container 2 for supplying a dosing quantity of the treatment medium from the container.

When the device is not in use, the cap 6 is placed on the mouth piece 4, in which (cap) then also the end 5.1 of the application tip 5 protruding over the mouth piece 4 is held tightly sealed.

For applying the treatment medium 8 to the treatment area, e.g. to the skin or tissue area to be treated, after removing the cap 6 and as applicable after actuating the dosing pump 3.1 or a corresponding dosing valve, the area to be treated is brushed over with the end 5.1 of the application tip 5. Due to the dosing pump 3.1 or due to the corresponding dosing valve this not only achieves the dosed supply of the treatment medium 8 from the interior of the container 2, but in particular also prevents the return of the treatment medium from the application tip 5 back into the interior of the container 2. This significantly improves the storage life of the treatment medium 8 in the container 2, especially also microbial contamination of the treatment medium 8 stored in the container 2. The applicator 1 allows the unproblematic, well dosed and also precisely localized application of the liquid treatment medium 8 on the area to be treated. The applicator according to the invention can therefore be used particularly, but not only, wherever a medicinal treatment with a liquid treatment medium is needed, particularly also in locally restricted area.

The applicator 1 is suitable for example for treating chronic wounds, such as Ulcus cruris or Decubitus, in which cases the treatment medium contains for example enzymatic and/or antimicrobial substances in a suitable solution and in the application of which the precise delimitation of the area to be treated from the adjacent tissue or the adjacent skin is at least very advantageous, to prevent toxic disturbances in the area of the wound which could make the skin susceptible to maceration.

The applicator 1 is furthermore suitable for applying a treatment medium to small wounds and scars, in particular also for all external applications of a medicinal substance which has to be applied in a very precise location, in particular also in the vicinity of critical areas, such as eyes, mucous membranes, etc.

The applicator 1 is furthermore suitable for application of solutions to the skin or the nails with substances for treating skin and nail mycosis disorders and their symptoms.

The applicator 1 is also suitable for application of solutions containing substances for treatment of warts and corns as well as bacterial and viral skin disorders.

In particular, the applicator 1, with a suitable treatment medium stored in the container 2, is suitable for treating calluses, warts, acne, herpes, onychomycosis, fungal infections, changes in pigment or pathological proliferations of the skin, etc.

Of special importance for the functioning ability of the applicator 1 and the result of the treatment, and also for the stability of the treatment medium, is the solution used, in which the substance required for the respective medicinal indication is contained.

Suitable solutions are for example oily solutions, which then, for example,

contain mineral oils such as paraffin oil, silicone oil and other their derivatives,

vegetable oils, such as sunflower seed oil,

oil products obtained from vegetable oils by ester interchange and/or partial synthesis, such as glycerol ester or other esters of fatty acids, also with fatty alcohols, fatty alcohols, their ethers and/or esters,

respectively as single components and/or in any combination and/or in a wide variety of single concentrations.

Such solutions can furthermore contain additives, such as tensides and/or waxes, the latter for thickening of the oil phase, fragrant oils and/or colorants. The tensides can then be contained in the solution for example in a concentration of approximately 0.1-4.0% by weight, and the waxes in a concentration of approximately 0.1-1.0% by weight, relative to the total weight of the solution respectively.

Further suitable solutions are aqueous alcoholic solutions. These solutions contain water (aqua dest.) and alcohols, for example monohydric and/or polyhydric alcohols, such as ethanol or isopropanol, namely for example in a concentration of approximately 5-70% by weight relative to the total weight of the solution, and/or polymer ethylene/propylene glycol PEC, and/or polyhydric saturated and unsaturated alcohols, such as propylene glycol and/or esters and ethers of alcohols in a concentration of for example 2-40% by weight relative to the total weight of the respective solution.

These alcoholic solutions can likewise contain further additives, in particular also for improving the stability and/or the physical properties and/or improving resorption of the respective solution and therefore of the treatment medium. Suitable additives for example are tensides for improving the solubility and surface spreading, such as polysobat 80, preferably in a concentration of 0.5-5.0% by weight, fatty alcohols, their esters and ethers, such as octyl dodecanol, preferably in a concentration of approximately 0.5-4.0% by weight, thickeners with a cellulose base, starch, oligosaccharide and/or polyacrylate, preferably in a concentration of approximately 0.1-0.4% by weight, fragrant oils and/or colorants. The above quantities specified in percent by weight likewise are relative to the total mass of the respective solution.

Especially in the case of the aqueous alcoholic solutions, it is also possible to provide the substances, particularly also those substances required for treatment of the respective indication and therefore for example the substances required for treatment of the above-mentioned indications, in liposomal and/or nanosomal encapsulation.

Further suitable solutions are aqueous solutions, in particular aqueous alcoholic solutions of the above-mentioned type, which additionally contain polymer components for film formation, i.e. for forming a polymer film covering the respective treatment area. These solutions then have a composition for example as specified above for the aqueous alcoholic solutions, but can also contain other solvents, such as ethyl acetate, preferably in a concentration of approximately 5.0-40% by weight, acetone, preferably in a concentration of approximately 5.0-40% by weight and or film breakers, such as other polymers and/or co- and cross-polymers, such as polymethacrylate (also chemically modified), which are suitable for forming elastic films on the treated area, for example on the skin, nail, pathologically changed tissue areas, such as warts, corns, etc.

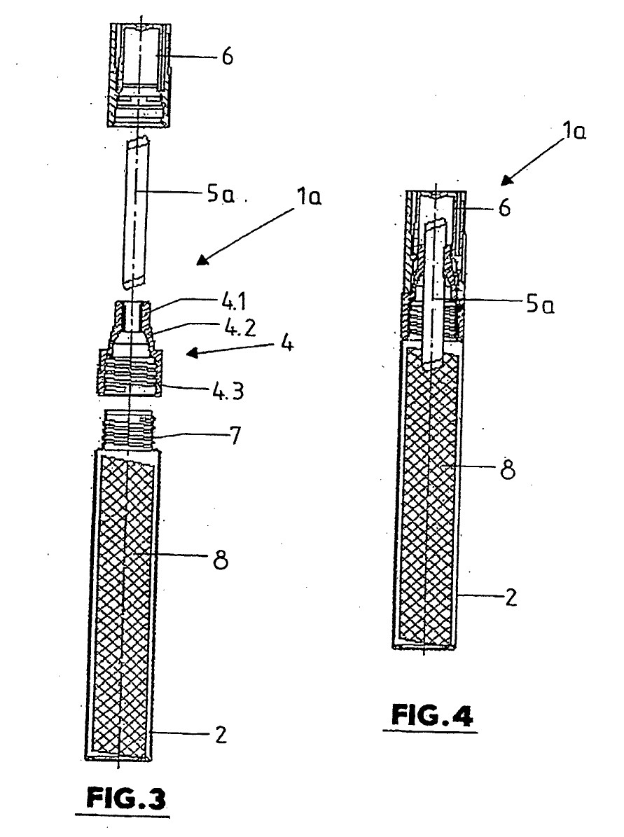

FIGS. 3 and 4 show a device 1a, which differs from the device 1 essentially only in that the insert 3 with the dosing pump or dosing valve is eliminated and the application tip 5a accordingly extends into the interior of the container or into the treatment medium 8 stored there. Otherwise the device 1a corresponds to the device 1, so that in FIGS. 3 and 4, the same reference numbers as those in FIGS. 1 and 2 are used for the elements to those of device 1. The device 1a is also suitable in particular for the treatment of the above-named indications using a treatment medium 8, which contains the substance or corresponding combination of substances required for treatment of the respective indication, namely likewise in an oily solution, an aqueous alcoholic solution and/or an aqueous or aqueous alcoholic solution with additional polymer components for forming a film.

The invention was described above based on exemplary embodiments. Numerous modifications and adaptations are possible without abandoning the underlying inventive idea upon which the invention is based.

For example it is possible to provide instead of the dosing pump 3.1 or instead of a dosing valve other dosing pumps and/or valve-like means for the dosed supply of the treatment medium and/or for preventing backflow of the treatment medium into the interior of the container 2 and/or for preventing dripping of the treatment medium. It was further assumed above that the container 2 is a tubular or sleeve-shaped container. Other forms are also conceivable; in particular, containers used to store the treatment medium can also be tube-shaped. To keep the application tip 5 at least on the outer surfaces of its end 5.1 protruding out of the mouth piece 4 as sterile as possible, it can be advantageous to provide means for disinfection in the cap, for example in the form of at least one cushion provided in the cap and saturated with disinfecting means, into which (cushion) the end 5.1 is immersed when placing the cap 6 on the device.

### REFERENCE LIST

1, 1a device or applicator
2 container
3 insert
3.1 dosing pump
4 mouth piece
4.1-4.3 sections of mouth piece 4
5, 5a application tip
5.1 application end of application tip
5.2 shank of application tip
6 cap
7 external thread
8 treatment medium or treatment solution with medicinal substance

What is claimed is:

1. A method for controlled application of a liquid medicinal treatment medium containing at least one medicinal substance on an area to be treated during an external treatment of a human or animal body, using a pen-like applicator, which holds the treatment medium in a reservoir or container and which is provided on an end with an application tip, by means of which the treatment medium from the interior of the container is applied to the area to be treated by brushing or moving over said area wherein the application tip is made of a porous material, a porous sintered material or a porous sintered polymer material.

2. The method according to claim 1, whereby the treatment medium contains at least one active substance in an oily solution.

3. The method according to claim 2, wherein the treatment medium oily solution contains at least one mineral oil or at least one derivative of a mineral oil.

4. The method according to claim 3, wherein mineral oil is paraffin oil or silicone oil.

5. The method according to claim 1, wherein the treatment medium solution contains at least one vegetable oil or an oil product which is obtained from at least one vegetable oil by ester interchange, partial reduction, etherification or partial synthesis.

6. The method according to claim 5, vegetable oil is sunflower oil.

7. The method according to claim 5, wherein the vegetable oil is glycerol ester or other esters of fatty acids with fatty alcohols, fatty alcohols or their ethers and esters.

8. The method according to claim 2, wherein the oily solution contains at least one additive in the form of tensides, with a content of approximately 0.1-4.0% by weight relative to the total weight of the solution, or in the form of waxes, with a content of approximately 0.1-1.0% by weight relative to the total weight of the solution, or fragrant oils or colorants.

9. The method according to claim 1, wherein the treatment medium contains at least one medicinal substance in an aqueous alcoholic solution.

10. The method according to claim 9, wherein the aqueous alcoholic solution contains monohydric or polyhydric alcohols, ethanol, glycerol or polymer alcohols or Isopropanol or propylene glycol or PEG or their esters or ethers.

11. The method according to claim 10, wherein the aqueous solution contains ethanol or isopropanol in a concentration of approximately 5.0-70% by weight relative to a total weight of the solution.

12. The method according to claim 10, wherein the aqueous alcoholic solution contains polyhydric, saturated or unsaturated alcohols and polymer alcohols, propylene glycol or their esters or ethers in a concentration of approximately 2.0-40% by weight relative to the total weight of the solution.

13. The method according to claim 1, wherein the use of an aqueous alcoholic solution contains at least one medicinal substance in liposomal or nanosomal encapsulation.

14. The method according to claim 1, wherein the aqueous alcoholic solution contains a film-forming component, or a polymer film-forming component.

15. The method according to claim 14, wherein the treatment medium further comprises ethyl acetate, in a concentration of approximately 5.0-40% by weight or acetone, in a concentration of approximately 5.0-40% by weight relative to the total weight of the solution, and is used as an additional solvent.

16. The method according to claim 14, wherein polymers or co- or cross-polymers, such as polymethacrylate, in chemically modified form, are used as the film-forming component.

17. The method according to claim 1, wherein the aqueous alcoholic solution contains additives in the form of tensides, in a concentration of approximately 0.5-5% by weight relative to the total weight of the solution, or fatty alcohols or their esters and ethers, in a concentration of approximately 0.5-4.0% by weight relative to a total weight of the solution.

18. The method according to claim 17, wherein the medium further comprises the use of polysobat 80 or octyl dodecanol as an additive.

19. The method according to claim 1, wherein the aqueous alcoholic solution contains additives in a form of thickeners based on cellulose or starch, oligosaccharide or polyacrylate, in a concentration of approximately 0.1-0.4% by weight relative to a total weight of the solution.

20. The method according to claim 1, wherein the solution contains fragrant oils or colorants.

21. The method according to claim 1, wherein the use of at least one medicinal substance is for treating calluses, warts, acne, herpes, onychomycosis, fungal infections on the head, changes in pigment or pathological proliferations of the skin.

22. The method according to claim 1, wherein the use of at least one medicinal substance is for treating chronic wounds, Ulcus cruris or Decubitus.

23. The method according to claim 22, wherein the treatment medium in the solution contains at least one enzymatic or antimicrobial substance.

24. (canceled)

25. The method according to claim 1, wherein an application tip has reduced hardness on an end used to apply a treatment medium to the skin.

26. The method according to claim 1, wherein the application tip exhibits reduced porosity on an end to apply a treatment medium to the skin.

27. The method according to claim 1, wherein the application tip is made of PE, PP, PVDF, PTFE or EVA.

28. A medicinal product with an applicator for controlled application of a liquid medicinal treatment medium containing at least one medicinal substance on an area to be treated during an external treatment of a human or animal body comprising a pen-like applicator, which holds the treatment medium in a reservoir or container and is provided on an end with an application tip, by means of which the treatment medium from an interior of the container is applied to an area to be treated by brushing over said area and the application tip made of a porous material, a porous sintered material or a sintered polymer material.

29. The medicinal product according to claim 28, wherein the container has a sleeve-shaped or tubular design.

30. The medicinal product according to claim 28, wherein the treatment medium contains at least one active substance in an oily solution.

31. The medicinal product according to claim 30, wherein the oily solution contains at least one mineral oil or at least one derivative of a mineral oil.

32. The medicinal product according to claim 30, wherein the mineral oil is paraffin oil or silicone oil.

33. The medicinal product according to claim 29, wherein the solution contains at least one vegetable oil or oil product which is obtained from at least one vegetable oil by ester interchange or partial synthesis.

34. The medicinal product according to claim 33, wherein sunflower seed oil is used as the vegetable oil.

35. The medicinal product according to claim 33, wherein glycerol ester or other esters of fatty acids with fatty alcohols, fatty alcohols or their ethers and esters are used as the oil product.

36. The medicinal product according to claim 28, wherein the oily solution contains at least one additive in the form of tensides, with a content of approximately 0.1-4.0% by weight relative to the total weight of the solution, or in the form of waxes, with a content of approximately 0.1-1.0% by weight relative to the total weight of the solution, or fragrant oils or colorants.

37. The medicinal product according to claim 28, wherein the treatment medium contains the at least one medicinal substance in an aqueous alcoholic solution.

38. The medicinal product according to claim 37, wherein the aqueous alcoholic solution contains monohydric or polyhydric alcohols, ethanol or isopropanol or propylene glycol or their esters or ethers.

39. The medicinal product according to claim 38, wherein the aqueous solution contains ethanol or isopropanol in a concentration of approximately 5.0-70% by weight relative to a total weight of the solution.

40. The medicinal product according to claim 38, wherein the aqueous alcoholic solution contains the polyhydric, saturated or unsaturated alcohols, propylene glycol or their esters or ethers in a concentration of approximately 2.0-40% by weight relative to a total weight of the solution.

41. The medicinal product according to claim 28, wherein the aqueous alcoholic solution contains at least one medicinal substance in liposomal or nanosomal encapsulation.

42. The medicinal product according to claim 28, wherein the aqueous alcoholic solution contains a film-forming component, or a polymer film-forming component.

43. The medicinal product according to claim 42, wherein ethyl acetate, in a concentration of approximately 5.0-40% by weight and/or acetone, in a concentration of approximately 5.0-40% by weight relative to a total weight of the solution, is used as a film-forming component.

44. The medicinal product according to claim 42, wherein polymers or cross-polymers, polymethacrylate, in chemically modified form, are used as the film-forming component.

45. The medicinal product according to claim 28, wherein the aqueous alcoholic solution contains additives in a form of tensides, in a concentration of approximately 0.5-5% by weight relative to a total weight of the solution, or fatty alcohols or their esters or ethers, in a concentration of approximately 0.5-4.0% by weight relative to a total weight of the solution.

46. The medicinal product according to claim 45, wherein polysobat 80 or octyl dodecanol are used as an additive.

47. The medicinal product according to claim 28, wherein the aqueous alcoholic solution contains additives in the form of thickeners based on cellulose or starch, oligosaccharide or polyacrylate, in a concentration of approximately 0.1-0.4% by weight relative to a total weight of the solution.

48. The medicinal product according to claim 28, wherein the solution contains fragrant oils or colorants.

49. (canceled)

50. (canceled)

51. (canceled)

52. (canceled)

53. (canceled)

54. (canceled)

55. (canceled)

56. A medicinal or medical product with an applicator for controlled application of a liquid medicinal treatment medium containing at least one medicinal substance on an area to be treated during an external treatment of a human or animal body, comprising
a pen-like applicator, which holds the treatment medium in a reservoir or container and which is provided on an end with an application tip, by means of which the treatment medium from an interior of the container is applied to the area to be treated by brushing or moving over said area, and at least one medicinal substance being a medicinal substance for treating warts.

57. The medicinal product according to claim 56, whereby the application tip is made of a porous material, a porous sintered material, or a sintered polymer material.

58. A method for controlled application of a liquid medicinal treatment medium containing at least one medicinal substance on an area to be treated during an external treatment of a human or animal body, using a pen-like applicator, which holds the treatment medium in a reservoir or container and which is provided on an end with an application tip, by means of which the treatment medium from the interior of the container is applied to the area to be treated by brushing or moving over said area, wherein the at least one medicinal substance is a medicinal substance for treating warts.

59. The method according to claim 58, wherein the application tip is made of a porous material, a porous sintered material, or a sintered polymer material.
